(19) United States
(12) Patent Application Publication
(10) Pub. No.: US 2009/0203958 A1
(43) Pub. Date: Aug. 13, 2009

(54) Apparatus and method for automatically inducing sleep deprivation in rodents

(76) Inventors: David Alan Johnson, Lawrence, KS (US); Erik Naylor, Lawrence, KS (US); Hans Patrick Harmon, Lawrence, KS (US); Seth Lawrence Gabbert, Lawrence, KS (US)

Correspondence Address: Pinnacle Technology, Inc., 2721 Oregon Street, Lawrence, KS, 66046, US

(21) Appl. No.: 12/069,146

(22) Filed: Feb. 7, 2008

(51) Int. Cl. A61M 21/00

(52) U.S. Cl. 600/26

(57) ABSTRACT

Apparatus and method for automatically inducing sleep deprivation in rodents with an electroencephalogram measurement and analysis system, an electromyogram measurement and analysis system, sleep deprivation stimulus, stimulus control, acquisition and real-time sleep stage analysis software, and cage with food and water supplies.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of 2R44MH076318-02 awarded by National Institute of Mental Health.

### CROSS REFERENCE APPLICATIONS

Not Applicable

### DESCRIPTION OF ATTACHED APPENDIX

Not Applicable

### BACKGROUND OF THE INVENTION

This invention relates generally to the field of neuroscience and more specifically to apparatus and method for automatically inducing sleep deprivation in rodents.

Sleep is a naturally occurring state in mammals which is characterized by four distinct behaviors: a specific sleeping posture, a decreased response to external stimuli, rapid reversibility and increased need following periods of deprivation; also referred to as the homeostatic load. While the exact cause of sleep remains unknown, it has been determined that sleep is a necessary function for life. Lack of sleep has been demonstrated to result in impairments in immune, endocrine, cognitive and behavioral function.

Sleep is defined in humans and other mammals by measurement of electroencephalograph (EEG) and electromyograph (EMG) waveforms. Measurable changes in these waveforms are hallmarks of the sleep process. Rodents have been shown to share many of the characteristics of human sleep including:
1) high frequency, low amplitude EEG waveforms predominately in the alpha (8-12 Hz) and beta (15-20 Hz) ranges during wakefulness
2) low frequency, high amplitude EEG waveforms in delta (0.5-4 Hz) range during non-rapid-eye-movement (NREM) sleep
3) low frequency, low amplitude EEG waveforms in the theta (4-7 Hz) range during rapid-eye-movement REM sleep.
   EMG waveforms undergo similar changes demonstrating high amplitude during waking and low, tonic activity during NREM and REM sleep.

There exists a need to selectively deprive an animal of sleep by application of a stimulus during times when the animal is determined to be asleep by measurement of EEG and EMG activity. A specific embodiment of the invention provides for this need by computerized monitoring of EEG and EMG activity in rodents and activation of a slowly rotating bar positioned at the bottom of the cage to gently push the animal until a wake state has occurred.

Arousal Technique: Electrical, vestibular or tactile stimulation—These techniques are very stressful to the animal and cannot maintain sleep deprivation indefinitely.
Platform Technique: The rodent is placed on a small platform above a water bath when muscle tone diminishes during rapid-eye movement sleep, the rodent makes contact with the water and wakes up. This technique confines the rodent to a very small space. The stress induced by this confinement may produce effects that would not be present with sleep deprivation alone. This technique does not allow periods of sleep deprivation and allowed sleep to be interspersed.
Multiple Platform Technique: This is the same as the platform technique, except multiple platforms are spaced far enough apart in a water path that the rodent cannot lay across multiple platforms to sleep, but some movement is allowed.
Pendulum Technique: A cage is suspended from a pendulum or rocked by some other means to create a constant imbalance that keeps the rodent from sleeping. This produces constant exercise as the animals move to the bottom of the cage to maintain balance.
Disk Technique: A rotating disk and water bath are used to create a situation in which the rodent is forced to walk in a direction opposite to disk rotation in order to stay out of the water. This technique also requires constant exercise during the sleep deprivation period.
Treadmill Technique: The rodent is forced to constantly walk on a treadmill or running wheel. This technique also requires constant exercise during the sleep deprivation period.

In order to study the process of sleep it is necessary to deprive an animal of sleep for either 1) a period of time longer than they would normally remain awake (acute) or 2) multiple extended periods over the course of days or weeks (chronic). At present, the mechanisms required to do this involve either a running treadmill or slowly rotating wheel apparatus capable of keeping the animal awake for prolonged periods by continuous exercise. While these mechanisms are successful in the short-term, exercise without a sufficient break remains a source of considerable stress on the animal and have been shown to adversely affect outcome measures. In particular, the beneficial effects of exercise can counteract the deleterious effects of sleep deprivation.

### BRIEF SUMMARY OF THE INVENTION

The primary object of the invention is to sleep deprive rodents.

Another object of the invention is exercise is not coerced.

Another object of the invention is a fully automated system.

Another object of the invention is comparison to a yoke controlled control rodent is not required.

A further object of the invention is configurable for simulation of shift work or other models of circadian rhythm sleep disorders such as advanced sleep phase or delayed sleep phase.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed apparatus and method for automatically sleep depriving rodents comprising: an electroencephalographic measurement and analysis system, an electromyographic measurement and analysis system, sleep deprivation stimulus, stimulus control, acquisition and real-time sleep stage analysis software, and cage.

In accordance with a preferred embodiment of the invention, there is disclosed a process for apparatus and method for automatically sleep depriving rodents comprising the steps of: an electroencephalographic measurement and analysis system, an electromyographic measurement and analysis system, sleep deprivation stimulus, stimulus control, acquisition and real-time sleep stage analysis software, and cage.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a schematic block diagram of the invention.

FIG. 2 is an exploded view of the invention.

FIG. 3 is a plan side view of the invention.

FIG. 4 is a plan top view of the invention.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

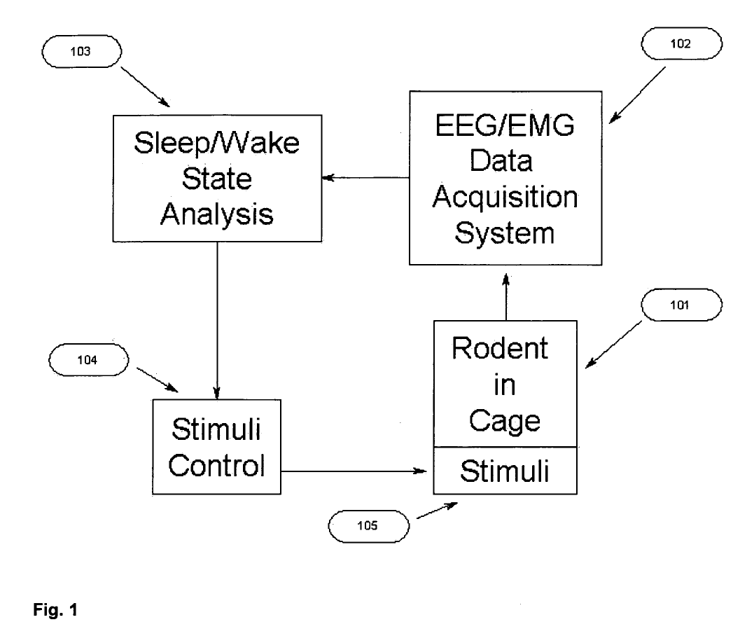

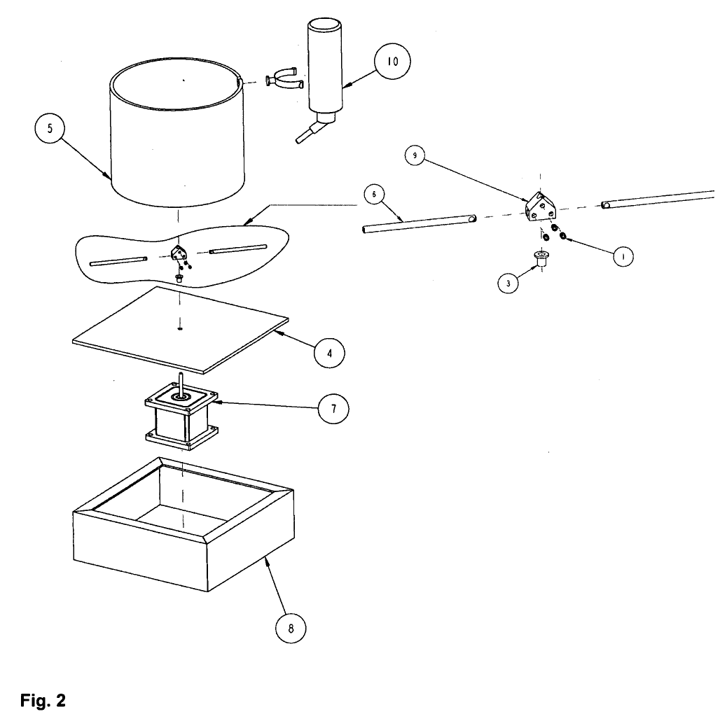

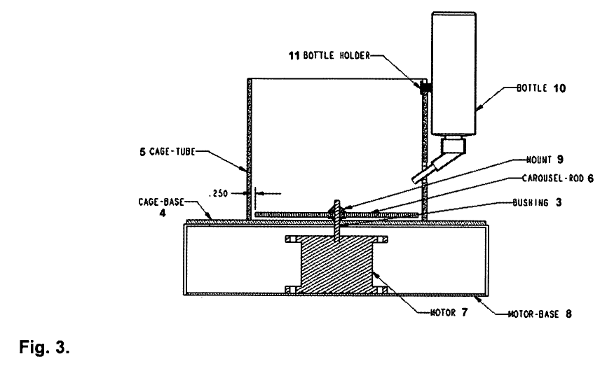

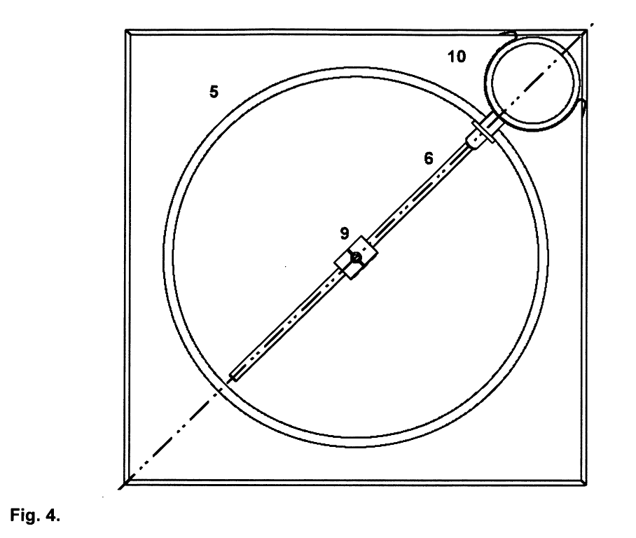

In accordance with the present invention, FIG. 1 shows a schematic overview of an apparatus and method for automatically inducing deprivation in rodents. A rodent in a properly designed enclosure 101 is monitored with a wired or wireless electroencephalography (EEG) and electromyography (EMG) system 102. The data collected in this manner are analyzed in real-time to determine whether the rodent is asleep or awake 103. The results of this analysis are used to activate or deactivate on or more stimuli 105 via a stimuli control 104. To accomplish an important function of the invention, there is shown in FIG. 2 an exploded view of a preferred embodiment of the stimuli and rodent cage. A motor 7 shaft in enclosure 8 extends through a ferrule 3 and the base of the rodent cage 4. The shaft is coupled 9 to a rotating bar 6. During wake periods the bar will be stationary. If sleep is detected via real time analysis of electromyographic and electroencephalographic data, the rotating bar will be activated. This forces the rodent to wake up in order to step over the bar; however, it does not force the animal to exercise. Simple control of the motor activation also allows long term models of shift work and other circadian rhythm sleep disorders. The cylindrical cage 5 walls confine the animal, and a water bottle 10 and optional food hopper are used to provide water and food. Turning to FIGS. 3 and 4, these are side and top plan views of the present invention that further illustrate the overall operation of the invention.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. Apparatus and method for automatically inducing sleep deprivation in rodents comprising:
an electroencephalographic measurement and analysis system;
an electromyographic measurement and analysis system;
sleep deprivation stimuli;
stimuli control;
acquisition and real-time sleep stage analysis software; and
cage with food and water supplies.

2. The Apparatus and method of claim 1, in which one of the sleep deprivation stimuli is a rotating bar.

3. A process for Apparatus and method for automatically inducing sleep deprivation in rodents comprising the steps of:
an electroencephalographic measurement and analysis system;
an electromyographic measurement and analysis system;
sleep deprivation stimuli;
stimuli control;
acquisition and real-time sleep stage analysis software; and
cage with food and water supplies.

4. The process for Apparatus and method of claim 3, in which one of the sleep deprivation stimuli is a rotating bar.
